(19) United States
(12) Patent Application Publication
(10) Pub. No.: US 2018/0161211 A1
(43) Pub. Date: Jun. 14, 2018

(54) Layered Therapy Bandage

(71) Applicant: Samuel Beckey, San Diego, CA (US)

(72) Inventor: Samuel Beckey, San Diego, CA (US)

(21) Appl. No.: 15/376,661

(22) Filed: Dec. 13, 2016

(51) Int. Cl. A61F 13/00; A61F 7/00; A61F 7/02; A61B 5/00; A61M 35/00

(52) U.S. Cl. CPC A61F 13/00063; A61F 7/007; A61F 7/02; A61F 13/00029; A61B 5/6802; A61F 2013/00217; A61F 2007/0075; A61F 2013/00285; A61F 2013/002; A61F 2013/0017; A61F 2013/0028; A61M 35/00

(57) ABSTRACT

The subject Device is an integrated therapy system in a bandage-like package optimized for specific medical conditions or diseases. The Device is designed around a layered package and delivers optimum medications and healing conditions to the skin or another membrane. The layers within the Device consist of passive or active components including medications, fluidic reservoirs and transport, dispersal pads, sensors, and electronic components, and these layers can be applied or not applied to the condition being addressed.

### CITATIONS

1. CN204337133U Wound surface treatment bandage for nursing,describes a bandage with lengthwise channel (groove) and “lateral fluid holes” along with a “drug reservoir bag” pierced by a lancet for drug delivery.
2. CN204147164U Physical Therapy Belt for Arteriovenous Internal Fistula, describes a elastic bandage fixing cold compress bags and a pair of inflatable pressurizing components that medical staff can activate.
3. CN203953945U Novel Bandage for Wound Healing describes a bandage with heating elements, heat conducting blocks, and a medicine storage with a blocking piece and cavity, allowing staff to add specific medicines.
4. CN204193326U Auxiliary Nursing Bandage for the Department of Burn describes an active sensor for temperature and a warming element using infrared LEDs, infrared spectra is claimed to irradiate deep tissues, kill bacteria, and repair cells.
5. WO2014146146A2 Light Therapy Bandage System describes a layered bandage secured by flexible fabric containing reflector, and spacer foam for “therapeutic lamps”.
6. U.S. Pat. No. 8,696,571B2 Continuity Circuits for Detecting Access Disconnection describes a bandage with detection of wetted blood surface through an electrical sensor made of a polymer that expands and breaks a circuit on absorption of liquid.
7. U.S. Pat. No. 7,687,678B2 Electronic Bandage with Flexible Electronic Controller describes a bandage with an attached controller (itself flexible) constructed to dynamically control application of treatment.
8. U.S. Pat. No. 6,589,270B2 Normothermic Treatment Apparatus with a Chemical, Phase Change, or Hot Water Means of Heating describes heater supported against tissue by a bandage with temperature maintained between 36 and 38 degrees C.
9. U.S. Pat. No. 5,578,022A Oxygen Producing Bandage and Method describes means to generate or reduce oxygen directly above a wound using at a gas permeable anode and an electrolyte.
10. CN2306025Y Medicinal Liquid Therapeutic Apparatus describes a bandage with a controller and cable controlling for temperature with an electrical heating plate, temperature sensor, along with a medicinal liquid in a spongy body.

### SUMMARY AND DESCRIPTION OF THE INVENTION

The described Layered Therapy Bandage is a system of therapy that can be optimized for specific conditions or diseases. It primarily addresses derma-pharmaceutical delivery and wound management, but can also be applied to other conditions including those described in the use cases. The Device seeks to maintain the ideal specified conditions to enable recovery, and can be configured and adopted to manage the immediate environment, deliver medications, monitor conditions, and adapt to changes during the course of treatment. It can also offer the ability to communicate its status through indications and wireless protocols so that the treatment can be monitored by the patient and the physician.

Layers within the Device include but are not limited to a selection of the following. Layers may be combined according to the needs of the therapy so that a combined active and passive layer is possible and, where space allows, multiple functions can occur on a single layer.
1. Cosmetic cover/Anti-Tampering layer
2. Gas permeable outer layer
3. Sealed outer layer
4. Temperature control layer with heating and/or cooling
5. Oxygen control layer with electrolyte and
6. Electrical or electronic layer with built-in micro-controller and power source
7. Cabled electrical layer for applications which require external electronics
8. Actuators incorporated in the electrical layers capable of operating on fluidic chambers
9. Fluidic layers with reservoirs for one or more medications
10. Time release or saturated medication layer
11. Pad or absorption layer for medication delivery
12. Non-stick permeable contact layer

Connections can be made between layers through the use of actuators (any of pressure, electrical, or chemical) and through protruding and folded connections as identified in the Description of Drawings section.

Usage Cases

In the usage cases below, the combination of the Device, the pre-loaded medications, and the electronic and software configuration is described as a “Pak”. Table 1 shows suggestions of typical medications that could be used in the Device.

TABLE 1

| | | One Time | | |
|---|---|---|---|---|
| | Oxygen | Delivery (and | | |
| | Permeable | Multitouch | Time Release | Continuous |
| | Bandage | Delivery) | Delivery | Antiseptic |
| Cuts with Scar | Yes (various | | Scar Reduction | Yes |
| Reduction | sizes) | | Cream | |
| Minor Cuts with | Yes | | | Yes |
| infection | | | | |
| prevention | | | | |
| Acne | Yes (round) | | Salicylic Acid | Benzoyl |
| | | | | Peroxide |
| Psoriasis | Yes (larger) | Capsaicin | Salicylic Acid | Corticosteroid |

Example Therapies Provided by the Device
A. Itch Pak: A simple and reduced example is the Itch Pak, which might consist of a mild anti-septic layer with a superimposed fluidic layer with manually dispensed anesthetic. One of more micro balloons of anti-septic could be dispensed by the user to reduce itching. No active or electronic components are necessary.
B. Scar Reduction Pak: A combination of antibiotics, antiseptics, and scar reduction medications can be delivered as a cocktail or over a time phased schedule.
C. Antiseptic: Where infection is a possibility, an active layer with sensors that could detect sepsis can be added to the traditional packing and bandage.
D. Acne: Using a slightly different form factor, the Device could be configured on small round
E. Psoriasis
F. Post Surgical Palo with wound drainage, followed by antibiotic dosing and, later, suture dissolving solvent (EtOH). These Palo could contain a bleeding detection electronic function.
G. Athletes feet Aerosol Bandage Pak (no need to breath in anti-fungal powder or pressurized in-the-can spray—just place the bandage on top of the toe junctions and push the compression button to release the Rx spheres).
H. In the field Trauma Pak (one series of pak could be a slap-on-your-arm bladder with a multiplicity of micro syringe needles capable of injecting drugs sub-dermally to suppress, shock, or reduce blood pressure, or stimulate breathing, mitigate a heart attack, prevent a seizure, as in the diabetic kit, etc.
I. Bite Pak: Multi-anti-sera immunoglobulin snakebite bandage, Dose and duration determined by the electronic layer.
J. Snorkeler's and Surfer's Pak (this kit would have various products with coagulants to stop cuts and bruises from coral, reefs, etc.+antibiotic)
K. The LifeGuard Station Pak , sting ray Pak, jellyfish Pak.
L. Insomnia up-dosing (one can hit the compression button every 5 minutes until the patient falls asleep)
M. Sea sickness/motion sickness up-dosing (same concept as above)
N. Drug succession Cocktail delivery
O Heroin overdose patch: Dose and duration based on physiological symptoms, manual setting, and/or electronic communication.
P. Viagra overdose pak (an example for a wide range of drugs where allergic reactions and/or over-dosing is a problem) that could be affixed and administered as soon as hypersensitive symptoms appear, especially true with antibiotics, blood thinners, etc. Benadryl injections are the preferred ER treatment when some shows up with hypersensitivity symptoms and/or anaphylaxis.
Q. Meth/PCP takedown Paks—this is a very high risk individual for law enforcement officers to encounter
R. Psychiatric Medications, such as Corrections calm down Paks (same concept as above for in-jail use)

### DESCRIPTION OF DRAWINGS

FIG. 1 shows an expanded view of the multi-layer structure, which could be laminated or otherwise secured on the edges to form the device. Callout 1 shows a wound or surface condition that the therapy will address. Item 2 identifies a skin surface, or other organ or the subject to which the device will attach. Item 3 shows a base layer described above that consists of a sterile, permeable, non-stick pad which may or may not have a sealed edge in contact with item 2.

Item 4 in FIG. 1 shows the top layer, which may be a cosmetic overlay, or may have various properties of elastic for compression, permeability for oxygen, or other characteristics. Item 5 shows the multiple layers, here there are three, used in the construction of the active components of the device.

FIG. 2 shows some possibilities for the active layers of the device. The layers can occur in any order as needed to construct the appropriate functions for the therapy device. Item 6 shows a traditional pad layer, which might also include antibiotic or disinfectant within that structure. It could be foam, a woven polyester, or an organic fiber, and might also be constructed from a gel polymer. Item 9 shows an active electronic circuit layer, with Item 7 being an electronic circuit, Item 8 being an onboard battery which itself could be a laminate or a small cell such as a hearing aid battery. Item 10 illustrates other active electronic components, such as a pressure actuator or a switch. Not the correspondence in location of the pressure actuator and the fluidic reservoir in Item 12.

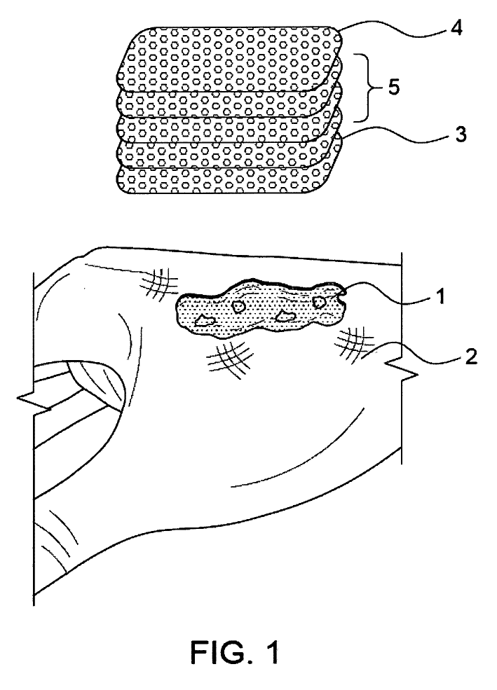

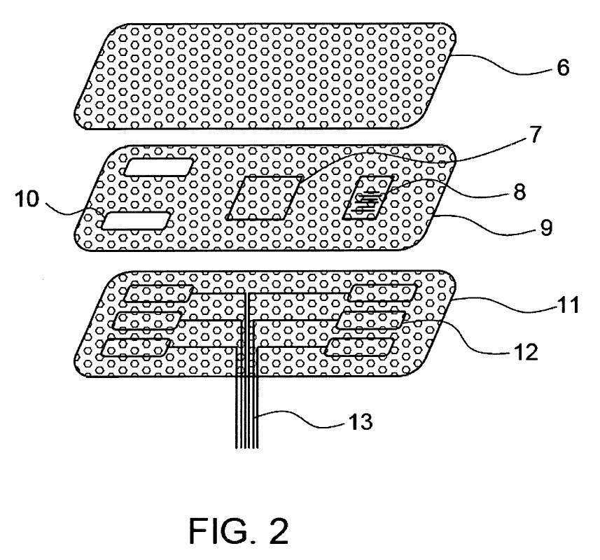

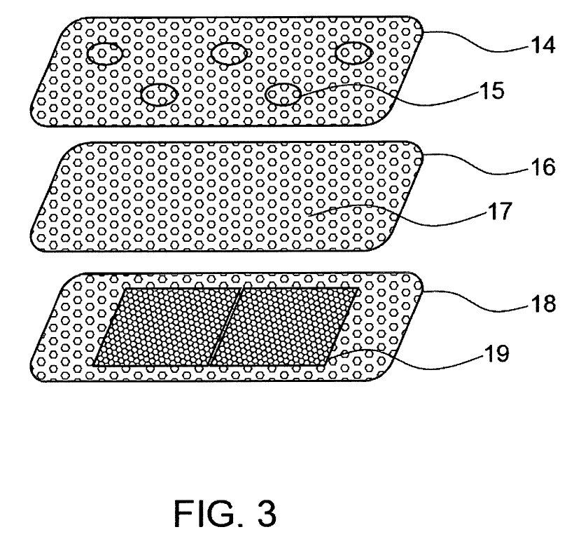

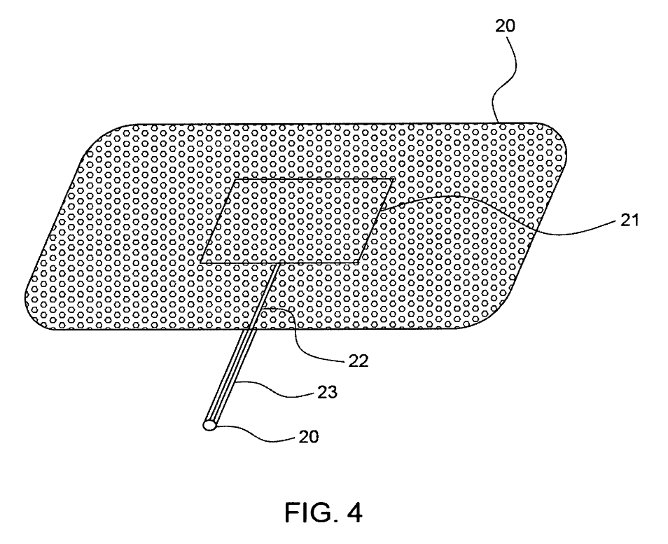

Item 11 in FIG. 2 shows an active layer containing in this case a plurality of fluid reservoirs 12. The fluid reservoirs can be associated with multiple medications and can be manually or electronically dispensed, or can be dispensed through capillary action as a time release mechanism. Fluid reservoirs can have a one time seal, such as by bursting micro balloons, a valve or other device, leading either to direct saturation of the layer below, or through the small capillary tubes indicated in Item 13. The tubes can be folded under other layers in the construction of the device, so that dispensing can be accomplished in any layer desired.

FIG. 3 shows addition and primarily passive layer possibilities. Item 14 is a series of sealed fluidic reservoirs that are evacuated through finger pressure, such as in the bug bite bandage usage case, although in this case five micro balloons are shown. In the bug bite bandage these would be single doses 15 of a mild anesthetic to reduce itching.

Item 16 in FIG. 3. shows a layer that would be pre-saturated with a continuous or time release medication. The medication would be specific to the specific indications for the condition being treated, as discussed in the usage cases above. Item 17 would be the saturated medication. Similarly, layers can be provided for Peltier heating and cooling. Temperature cycling is indicated for all types of orthopedic surgeries, dental surgeries, sprains, soft tissue injuries, etc.

Item 18 in FIG. 3 shows an encapsulating honeycomb structure material that can be used to both deliver very small doses of medication in highly targeted areas, can benefit time release of medications according to the honeycomb 19 size, and can respond to mechanical pressure, again either manually or with electronic or pressure actuators, release medication on command.

FIG. 4 expands on the layered delivery technique by adding electronic sensors as well as fluidic operations. Sensors including but not limited to temperature, humidity, liquid detection, oxygenation, vital signs, and pressure can be supplied and positions as needed for therapeutic effect, combining with the electronic capability shown in Item 9. A sensor equipped layer 20 contains electronic circuitry 21 which may or may not be mechanically flexible with the layer. Flexible wiring 22 connections to a protrusion from the layer, which can be wrapped or folded to place the active sensor in the desired location on the device. Finally, a sensor 24 is located at the end of the flexible wiring for the above functions. The combination of the electrical layer disclosed here with a fluidic or fluid saturated layer can be used to deliver oxygen through the use of an electrode (anode) and electrolyte, thereby allowing oxygen level to be measured and manipulated for therapeutic effect.

What is claime is:

1. A drug delivery and healing Device using a multilayered construction similar to a bandage that is configured and/or constructed for specific medical indications, comprising:
a. Layers with either or both active and passive properties
b. Delivery of medication specific to the condition being treated
c. A means to contain the medication to the active site

2. The Device in claim 1, where additionally the control of the healing environment is maintained in terms of any environmental factor, including but not limited to:
a. Temperature control which may include both heating and cooling
b. Oxygen control which may include both increasing or decreasing oxygenation
c. Pressure control
d. Humidity control

3. The Device described in claim 1, where active layers can include one or more electronic and electrical layers including functions such as logic, timers, controllers, dispensers, actuators, and communications.

4. The Device described in claim 1, where an active electronic layer may contain sensors, including but not limited to:
a. Patient vital signs
b. Environmental factors necessary for closed loop control of the items in claim 2.

5. The DeviCe described in claim 1, where layers may contain one or more reservoirs for medications which can be dispensed actively (electronically) or manually.

6. The Device described in claim 1, where an electronic layer can communicate with other devices for either or both of the following:
a. Recording and logging patient condition and treatment results
b. Configuring or adjusting the functions of the Device.

7. The Device described in claim 1, where layers can include, but are not limited to:
a. Dermal contact layer with non-stick and antiseptic properties offering an optional seal around the treatment area.
b. Continuous medication layer with or without impregnated medications such as an antibiotic and which also forms a pad for the delivery of additional medications by other layers.
c. Fluidic reservoir layers
d. Time release medication layers, using either active or passive techniques
e. Active electronic or electrical layer
f. Environmental control layers

8. The Device described in claim 1 for uses other than transdermal medical delivery.
